(19) United States
(12) Patent Application Publication
(10) Pub. No.: US 2025/0075183 A1
(43) Pub. Date: Mar. 6, 2025

(54) METHOD FOR CO-CULTIVATION AND DIRECTED DIFFERENTIATION INDUCTION OF MUSCLE SATELLITE CELLS AND ADIPOSE-DERIVED STEM CELLS

(71) Applicants: OCEAN UNIVERSITY OF CHINA, Qingdao (CN); QINGDAO INSTITUTE OF MARINE BIORESOURCES FOR NUTRITION & HEALTH INNOVATION, Qingdao (CN)

(72) Inventors: Changhu XUE, Qingdao (CN); Hongwei ZHENG, Qingdao (CN); Xuan ZHOU, Qingdao (CN); Yingxin WEI, Qingdao (CN); Haowen YIN, Qingdao (CN)

(73) Assignees: OCEAN UNIVERSITY OF CHINA, Qingdao (CN); QINGDAO INSTITUTE OF MARINE BIORESOURCES FOR NUTRITION & HEALTH INNOVATION, Qingdao (CN)

(21) Appl. No.: 18/813,100

(22) Filed: Aug. 23, 2024

(30) Foreign Application Priority Data
Aug. 28, 2023 (CN) 202311090006.8

(51) Int. Cl. C12N 5/077; C12N 5/0775

(52) U.S. Cl. CPC C12N 5/0659; C12N 5/0667; C12N 2501/999; C12N 2502/1335; C12N 2502/1382; C12N 2506/1323; C12N 2506/1384; C12N 2513/00

(57) ABSTRACT

A method for co-cultivation and directed differentiation induction of muscle satellite cells and adipose-derived stem cells is provided. The method includes the steps of co-cultivation and co-differentiation. The method for co-cultivation and directed differentiation induction of muscle satellite cells and adipose-derived stem cells provided by the present disclosure can allow the effective co-cultivation and co-directed differentiation induction of muscle satellite cells and adipose-derived stem cells from Larimichthys crocea, thereby providing a feasible solution for the large-scale production of high-quality cultivated meat.

### CROSS REFERENCE TO THE RELATED APPLICATIONS

This application is based upon and claims priority to Chinese Patent Application No. 202311090006.8, filed on Aug. 28, 2023, the entire contents of which are incorporated herein by reference.

### TECHNICAL FIELD

The present disclosure belongs to the technical field of cultivation of meat with stem cells and animal cells, and specifically relates to a method for co-cultivation and directed differentiation induction of muscle satellite cells and adipose-derived stem cells.

### BACKGROUND

Traditional meat production is faced with problems such as resource shortages, environmental pollution, and animal welfare. Research on cultivated meat has become one of the important ways to solve these problems. Larimichthys crocea, a high-quality edible fish species, has delicious meat and is rich in proteins and fats. Thus, Larimichthys crocea is very suitable for the production of cell-cultivated meat.

Muscle satellite cells are myogenic precursor cells or unipotent myogenic stem cells with proliferative and self-renewal abilities in muscle tissues. Muscle satellite cells have a prominent differentiation potential and can develop into functional muscle cells. Adipose-derived stem cells have a strong adipogenic differentiation ability and thus, can provide an improved texture and taste for cultivated meat. Therefore, the co-cultivation and directed differentiation induction of muscle satellite cells and adipose-derived stem cells from Larimichthys crocea is the key to improving the quality and taste of cultivated meat.

In most of the current cultivation methods, muscle tissue and adipose tissue are cultivated and differentiated separately. However, the actual animal meat is mostly a mixture of muscle and adipose tissues. Therefore, the product obtained by a separate cultivation method still has a different taste from the actual animal meat to some extent. There is still a lack of an effective method to allow the co-cultivation and directed differentiation induction of muscle satellite cells and adipose-derived stem cells from Larimichthys crocea.

### SUMMARY

The technical problem to be solved by the present disclosure is that there is still a lack of an effective method to allow the co-cultivation and directed differentiation induction of muscle satellite cells and adipose-derived stem cells from Larimichthys crocea in the research on cell-cultivated meat.

In order to solve the above problem, the present disclosure provides a method for co-cultivation and directed differentiation induction of muscle satellite cells and adipose-derived stem cells, which can allow the effective co-cultivation and co-directed differentiation induction of muscle satellite cells and adipose-derived stem cells from Larimichthys crocea, thereby providing a feasible solution for the large-scale production of high-quality cultivated meat.

In order to allow the above objective, the present disclosure adopts the following technical solutions: A method for co-cultivation and directed differentiation induction of muscle satellite cells and adipose-derived stem cells is provided, including the following steps:
(1) co-cultivation: resuspending muscle satellite cells and adipose-derived stem cells from Larimichthys crocea with a Larimichthys crocea stem cell complete medium, mixing in any ratio, plating, and cultivating in a biochemical incubator at 26° C. to 30° C., where the medium is changed every two days; and
(2) co-differentiation: when the muscle satellite cells and the adipose-derived stem cells from the Larimichthys crocea co-cultivated in the step (1) grow to a density of 90% or more, removing the Larimichthys crocea stem cell complete medium, adding a Larimichthys crocea adipogenic differentiation medium to allow differentiation for 7 d to 21 d, where the Larimichthys crocea adipogenic differentiation medium is changed every other day, and adding a Larimichthys crocea myogenic differentiation medium to allow myogenic differentiation for 4 d to 6 d, where the co-differentiation lasts for 11 d to 27 d in total. The adipogenic differentiation is first induced due to the following reason: The Larimichthys crocea adipogenic differentiation medium can maintain the basic growth conditions for the muscle satellite cells from the Larimichthys crocea while inducing the adipogenic differentiation of the adipose-derived stem cells from the Larimichthys crocea. The myogenic differentiation is induced after the adipogenic differentiation is induced, such that the muscle satellite cells from the Larimichthys crocea can undergo myogenic differentiation without significantly affecting an adipogenic differentiation process of the adipose-derived stem cells from the Larimichthys crocea. In this way, the respective differentiation of the muscle satellite cells and the adipose-derived stem cells from the Larimichthys crocea can be induced in a same environment.

Further, in the step (1), the Larimichthys crocea stem cell complete medium is 90% DMEM/F12 basal medium+10% fetal bovine serum. The complete medium can meet the nutritional requirements of normal growth of both the muscle satellite cells and the adipose-derived stem cells from Larimichthys crocea.

Further, in the step (2), the Larimichthys crocea adipogenic differentiation medium includes 90% DMEM/F12 basal medium+10% fetal bovine serum, and further includes the following adipogenic induction factors: insulin at a concentration of 1 mg/L to 10 mg/L, dexamethasone at a concentration of 0.5 μmol/L to 5 μmol/L, rosiglitazone at a concentration of 2 μmol/L to 20 μmol/L, indomethacin at a concentration of 10 μmol/L to 100 μmol/L, 3-isobutyl-1-methylxanthine at a concentration of 0.05 mmol/L to 1 mmol/L, arachidonic acid at a concentration of 1 μg/mL to 20 μg/mL, cholesterol at a concentration of 110 μg/L to 2,200 μg/L, tocopheryl acetate at a concentration of 35 μg/L to 700 μg/L, oleic acid at a concentration of 5 μg/L to 100 μg/L, linoleic acid at a concentration of 5 μg/L to 100 μg/L, palmitic acid at a concentration of 5 μg/L to 100 μg/L, stearic acid at a concentration of 5 μg/L to 100 μg/L, linolenic acid at a concentration of 5 μg/L to 100 μg/L, and myristic acid at a concentration of 5 μg/L to 100 μg/L.

Arachidonic acid: Arachidonic acid is a polyunsaturated fatty acid involved in the regulation of growth and development of adipocytes. Arachidonic acid can synthesize a series of signaling molecules that promote the proliferation and differentiation of adipocytes, and can affect the number and size of adipocytes.

Cholesterol: Cholesterol is an important lipid component that plays a key role in the adipogenic differentiation of adipocytes. Cholesterol can promote the production of adipocytes and participate in the establishment and stabilization of cell membranes.

Tocopheryl acetate: Tocopheryl acetate is a form of vitamin E, and has antioxidant and anti-inflammatory effects. Tocopheryl acetate can protect adipocytes from an oxidative stress while promoting the differentiation and functional maturation of adipocytes.

Oleic acid: Oleic acid is a monounsaturated fatty acid common in vegetable oils and animal fats. During the adipogenic differentiation of adipocytes, oleic acid is involved in the synthesis and storage of triacylglycerol while affecting the metabolism and function of adipocytes.

Linoleic acid: Linoleic acid is a polyunsaturated fatty acid, and is one of the essential fatty acids in the human body. During the adipogenic differentiation of adipocytes, linoleic acid is involved in the construction and stabilization of cell membranes and regulates the growth and development of adipocytes.

Palmitic acid: Palmitic acid is a saturated fatty acid common in natural oils. Palmitic acid is involved in the regulation of differentiation and metabolism of adipocytes, and affects the regulation of fatty acid synthesis and adipocyte size and number.

Stearic acid: Stearic acid is a saturated fatty acid common in animal fats. During an adipogenic process of adipocytes, stearic acid is involved in the synthesis and storage of triacylglycerol while affecting the metabolism and function of adipocytes.

Linolenic acid: Linolenic acid is a polyunsaturated fatty acid, and is one of the essential fatty acids in the human body. During the adipogenic differentiation of adipocytes, linolenic acid regulates the fluidity and stability of cell membranes and promotes the differentiation and development of cells.

Myristic acid: Myristic acid is a saturated fatty acid found in vegetable oils and animal fats. Myristic acid is involved in the differentiation and maturation of adipocytes, and affects the size, metabolism, and function of cells.

Further, in the step (2), the Larimichthys crocea myogenic differentiation medium includes 98% DMEM/F12 basal medium+2% horse serum, and further includes the following myogenic induction factor: vitamin D at a concentration of 10 ng/ml to 300 ng/mL.

Further, in the Larimichthys crocea stem cell complete medium and the Larimichthys crocea adipogenic differentiation medium, antibiotics are further added. As a specific record of the embodiment, 1.0×10⁵ U/L of benzylpenicillin sodium, 100 mg/L of streptomycin, and 2.5 mg/L of amphotericin B are added.

Further, in the step (1), a process for isolating the muscle satellite cells and the adipose-derived stem cells from the Larimichthys crocea includes the following steps:
(0-1) collecting a muscle tissue or an adipose tissue from the Larimichthys crocea, cutting the muscle tissue or the adipose tissue into pieces, and adding a digestion solution to allow digestion for 0.2 h to 3 h to obtain a digested tissue mixed solution; adding a washing solution to the digested tissue mixed solution for washing, filtering to obtain a cell suspension, and centrifuging the cell suspension at 800 rpm/min to 2,000 rpm/min for 5 min to 10 min to collect cells; and adding the washing solution to the cells for washing, and centrifuging to collect cells,
where a mass-to-volume ratio of the muscle tissue or the adipose tissue to the digestion solution is 1:1 to 1:10, a mass-to-volume ratio of the muscle tissue or the adipose tissue to the washing solution is 1:1 to 1:10, a formula of the digestion solution includes 0.1 mg/mL to 1 mg/mL of collagenase type I and 0.05 mg/mL to 1 mg/mL of trypsin, and the washing solution is a D-hanks solution;
(0-2) if visible red blood cells are observed in a cell pellet produced after the centrifugation, conducting red blood cell lysis; and if no visible red blood cells are observed in the cell pellet produced after the centrifugation, directly proceeding to the next step; and
(0-3) resuspending cells with the Larimichthys crocea stem cell complete medium to obtain a cell suspension, inoculating the cell suspension in a 6-well cell culture plate, and cultivating for 3 h to obtain a cell solution; transferring the cell solution to a new well, supplementing the Larimichthys crocea stem cell complete medium to 3 mL, and cultivating at 28° C.; and when a cell confluency reaches 85% to 90%, conducting passage.

Further, the passage is conducted as follows: removing an old medium, adding 1 mL to 3 mL of phosphate-buffered saline (PBS) to wash cells 1 to 2 times to remove the residual serum, and adding 100 μL to 300 μL of 0.25% trypsin to digest the cells; when it is observed under a microscope that the cells become spherical and most of the cells fall off after tapping, adding 1 mL of a Larimichthys crocea stem cell complete medium to stop the digestion, transferring a cell suspension into a 2 mL Eppendorf tube and centrifuging the cell suspension at 1200 rpm for 5 min, adding 2 mL of the Larimichthys crocea stem cell complete medium to resuspend the cells, and gently pipetting up and down several times to obtain a cell suspension; dispensing the cell suspension into new T25 flasks according to a volume ratio of 1:2 or 1:3, and supplementing a medium system in each T25 flask to 5 mL; and sealing and labeling the T25 flasks, and incubating in a biochemical incubator at 28° C.

The present disclosure has the following beneficial effects:
(1) The present disclosure establishes a key technical system for isolation-preparation, cultivation, passage, and differentiation of muscle satellite cells and adipose-derived stem cells from Larimichthys crocea, including operation steps and methods for the establishment and passage of muscle satellite cell and adipose-derived stem cell lines from Larimichthys crocea and compositions of media, a digestion solution, and a washing solution. As a result, the present disclosure lays a prominent foundation for the large-scale production of cell-cultivated meat and saves the corresponding costs.
(2) The use of the method for co-cultivation and co-differentiation of muscle satellite cells and adipose-derived stem cells from Larimichthys crocea provided by the present disclosure can successfully induce the in vitro myogenic and adipogenic differentiation of muscle satellite cells and adipose-derived stem cells from Larimichthys crocea respectively, and allow the simultaneous cultivation, proliferation, and differentiation of muscle satellite cells and adipose-derived stem cells from large yellow croaker while making the two not significantly affect each other, which solves the problem that the existing cell-cultivated fish meat has a single component. Through the co-cultivation and co-differentiation of muscle satellite cells and adipose-derived stem cells from Larimichthys crocea, an interaction between these two cells can be promoted to allow a synergistic effect, such that cultivated meat has a close texture to an actual muscle tissue, which further improves a taste and flavor of the cultivated meat, allows the cell-cultivated meat to have balanced and healthy nutritional components, and meets the requirements of consumers for nutritional needs.

### BRIEF DESCRIPTION OF THE DRAWINGS

FIG. 1 is an image of muscle satellite cells from Larimichthys crocea;

FIG. 2 is an image of adipose-derived stem cells from Larimichthys crocea;

FIG. 3 shows oil red O staining images of muscle satellite cells and adipose-derived stem cells from Larimichthys crocea after co-cultivation and co-differentiation, where each ratio refers to a ratio of a number of muscle satellite cells from Larimichthys crocea to a number of adipose-derived stem cells from Larimichthys crocea during inoculation;

FIG. 4 shows immunofluorescence staining images of muscle satellite cells and adipose-derived stem cells from Larimichthys crocea after co-cultivation and co-differentiation, where cell lipids are stained into a bright color, cell nuclei are stained into a dark color, and each ratio refers to a ratio of a number of muscle satellite cells from Larimichthys crocea to a number of adipose-derived stem cells from Larimichthys crocea during inoculation; and

FIG. 5 shows immunofluorescence staining images of muscle satellite cells and adipose-derived stem cells from Larimichthys crocea after co-cultivation and co-differentiation, where MHCs are stained into a dark color (tubular zones), cell nuclei are stained into a dark color (punctate zones), and each ratio refers to a ratio of a number of muscle satellite cells from Larimichthys crocea to a number of adipose-derived stem cells from Larimichthys crocea during inoculation.

### DETAILED DESCRIPTION OF THE EMBODIMENTS

In order to make the objectives, technical solutions, and advantages of the embodiments of the present disclosure clear, the technical solutions in the embodiments of the present disclosure are described clearly and completely below. Apparently, the described embodiments are some rather than all of the embodiments of the present disclosure. All other embodiments obtained by those skilled in the art based on the embodiments of the present disclosure without creative efforts shall fall within the protection scope of the present disclosure.

### Example 1 Isolation-Preparation and Cultivation of Muscle Satellite Cells and Adipose-Derived Stem Cells from Larimichthys crocea

A muscle tissue and an adipose tissue were collected from the Larimichthys crocea and cut into pieces, and a digestion solution was added to allow digestion for 1 h to obtain a digested tissue mixed solution. A washing solution was added to the digested tissue mixed solution for washing to obtain a mixed solution, the mixed solution was filtered to obtain a cell suspension, and the cell suspension was centrifuged at 1,500 rpm/min for 10 min to collect cells. The washing solution was added to the cells for washing to obtain a cell solution, and the cell solution was centrifuged at 1,500 rpm/min for 10 min to collect cells.

A ratio of the muscle tissue and the adipose tissue to the digestion solution was 1:5, a ratio of the muscle tissue and the adipose tissue to the washing solution was 1:5, a formula of the digestion solution included 0.25 mg/mL of collagenase type I and 0.25 mg/mL of trypsin, and the washing solution was a D-hanks solution.

The cells were resuspended with the Larimichthys crocea stem cell complete medium to obtain a cell suspension. The cell suspension was inoculated in a 6-well cell culture plate, cultivated for 3 h, and then transferred to a new well, the Larimichthys crocea stem cell complete medium was supplemented to 3 mL, and the plate was incubated at 28° C. When a cell confluency reached 90%, passage was conducted. The passage was conducted as follows: An old medium was removed, 3 mL of PBS was added to wash cells 1 time to remove the residual serum, and 300 μL of 0.25% trypsin was added to digest the cells. When it was observed under a microscope that the cells became spherical and most of the cells fell off after tapping, 1 mL of a Larimichthys crocea stem cell complete medium was added to stop the digestion, a cell suspension was transferred into a 2 mL Eppendorf tube and centrifuged at 1200 rpm for 5 min, then 2 mL of the Larimichthys crocea stem cell complete medium was added for resuspending the cells to obtain a mixed system, and the mixed system was gently pipetted up and down several times to obtain a cell suspension. The cell suspension was dispensed into new T25 flasks according to a ratio of 1:2, and a medium system in each T25 flask was supplemented to 5 mL. The T25 flasks were sealed, labeled, and incubated in a biochemical incubator at 28° C.

A formula of the Larimichthys crocea stem cell complete medium was 90% DMEM/F12 basal medium+10% fetal bovine serum. Further, in the Larimichthys crocea stem cell complete medium, antibiotics were also added, and as a specific record of the embodiment, 1.0×10⁵ U/L of benzylpenicillin sodium, 100 mg/L of streptomycin, and 2.5 mg/L of amphotericin B were added.

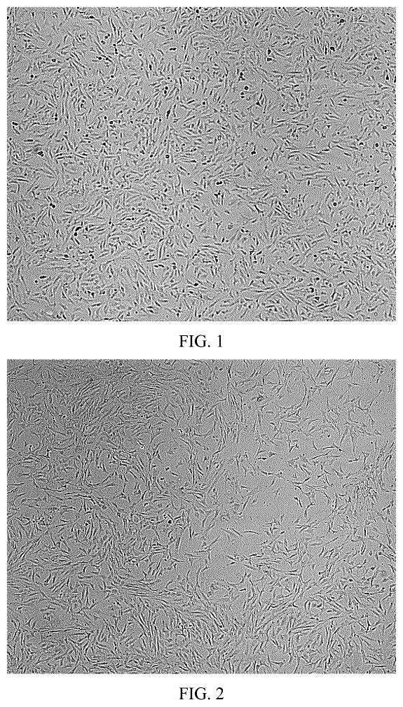

According to the results in FIG. 1 and FIG. 2, the muscle satellite cells from Larimichthys crocea and the adipose-derived stem cells from Larimichthys crocea were in prominent growth statuses.

### Example 2 Co-Cultivation and Co-Differentiation of Muscle Satellite Cells and Adipose-Derived Stem Cells from Larimichthys crocea

Muscle satellite cells and adipose-derived stem cells from Larimichthys crocea each were resuspended with a Larimichthys crocea stem cell complete medium, mixed according to ratios of 4:6, 5:5, 6:4, 7:3, 8:2, and 9:1, plated, and cultivated in a biochemical incubator at 28° C., where the medium was changed every two days. When the muscle satellite cells and the adipose-derived stem cells from Larimichthys crocea co-cultivated grew to a density of 90% or more, the Larimichthys crocea stem cell complete medium was removed, a Larimichthys crocea adipogenic differentiation medium was added to allow differentiation for 14 d, where the Larimichthys crocea adipogenic differentiation medium was changed every other day, and then a Larimichthys crocea myogenic differentiation medium was added to allow myogenic differentiation for 6 d. The co-differentiation lasted for 20 d in total.

A formula for the Larimichthys crocea adipogenic differentiation medium was 90% DMEM/F12 basal medium+10% fetal bovine serum, and on this basis, the following adipogenic induction factors were added: insulin at a concentration of 5 mg/L, dexamethasone at a concentration of 1 μmol/L, rosiglitazone at a concentration of 2 μmol/L, indomethacin at a concentration of 10 μmol/L, 3-isobutyl-1-methylxanthine at a concentration of 0.1 mmol/L, arachidonic acid at a concentration of 2 μg/mL, cholesterol at a concentration of 110 μg/L, tocopheryl acetate at a concentration of 50 μg/L, oleic acid at a concentration of 10 μg/L, linoleic acid at a concentration of 10 μg/L, palmitic acid at a concentration of 10 μg/L, stearic acid at a concentration of 10 μg/L, linolenic acid at a concentration of 10 μg/L, and myristic acid at a concentration of 10 μg/L. Antibiotics: 1.0×10⁵ U/L of benzylpenicillin sodium, 100 mg/L of streptomycin, and 2.5 mg/L of amphotericin B.

A formula for the Larimichthys crocea myogenic differentiation medium was 98% DMEM/F12 basal medium+2% horse serum, and on this basis, the following myogenic induction factor was added: vitamin D at a concentration of 50 ng/mL.

The co-differentiation of the muscle satellite cells and adipose-derived stem cells from Larimichthys crocea was conducted in a biochemical incubator at 28° C.

Myogenic and adipogenic differentiation effects of the muscle satellite cells and adipose-derived stem cells were detected by an immunofluorescence staining method. A specific process was as follows:
1) MHC immunofluorescence expression:

After the co-cultivation of muscle satellite cells and adipose-derived stem cells from Larimichthys crocea was completed, the old medium was removed and a cell monolayer was washed with PBS. Then 4.0% paraformaldehyde was added to allow fixation at room temperature for 10 min, and the cell monolayer was washed with PBS. 0.1% Triton X-100 was added to permeabilize cells at room temperature for 5 min, and then the cell monolayer was washed with PBS. A 10% bovine serum albumin (BSA) blocking solution was added to block for 3 h, and washing was conducted 3 times with PBS. Mouse anti-MHC was added at 1:100, incubation was conducted overnight at 4° C., and washing was conducted 3 times with PBS. A PE-labeled goat anti-mouse secondary antibody was added at 1:100, incubation was conducted at room temperature for 1 h, and washing was conducted 3 times with PBS. 4′,6-diamidino-2-phenylindole (DAPI) was added, and incubation was conducted at room temperature for 15 min. A fluorescence signal was photographed and recorded under an inverted fluorescence microscope.
2) Adipogenic differentiation detection:

After the co-cultivation of muscle satellite cells and adipose-derived stem cells from Larimichthys crocea was completed, the old medium was removed and a cell monolayer was washed with PBS. Then 4.0% paraformaldehyde was added to allow fixation at room temperature for 10 min, and the cell monolayer was washed with PBS. 0.1% Triton X-100 was added to permeabilize cells at room temperature for 5 min, and then the cell monolayer was washed with PBS. A Nile Red staining solution was added to stain cells for 30 min, and then the cell monolayer was washed with PBS. DAPI was added to stain cell nuclei for 5 min, and then the cell monolayer was washed with PBS. PBS was added, and a fluorescence signal was photographed and recorded under an inverted fluorescence microscope.

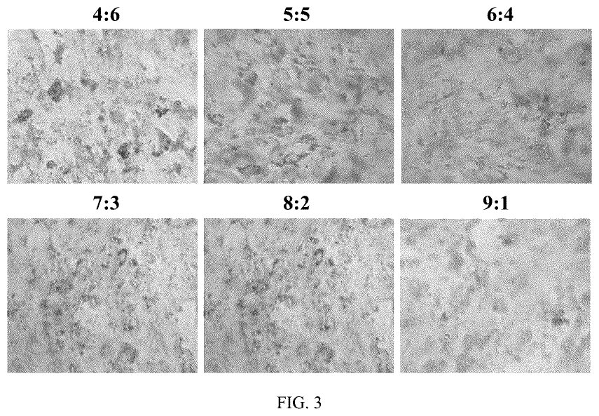

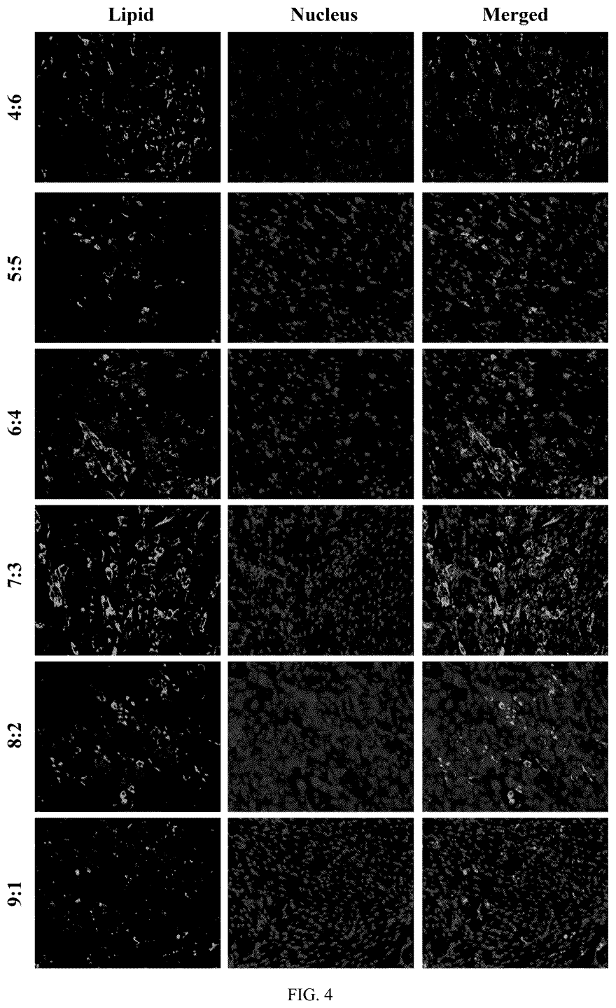

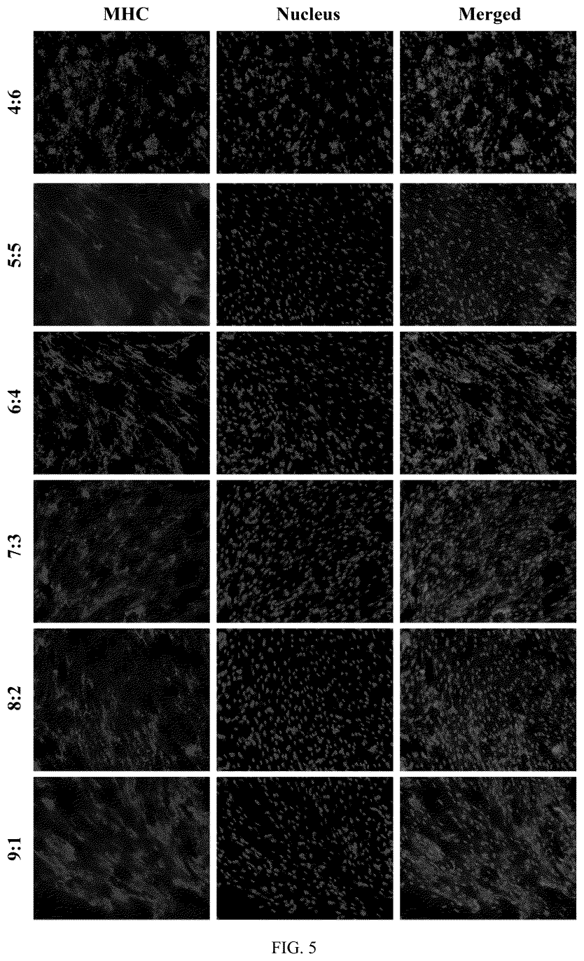

According to results in FIG. 3 and FIG. 4, after the co-cultivation of muscle satellite cells and adipose-derived stem cells from Larimichthys crocea in different ratios was completed, it was observed that lipids were accumulated in large quantities in the cells, indicating that the adipose-derived stem cells underwent excellent adipogenic differentiation.

According to results in FIG. 5, after the co-cultivation of muscle satellite cells and adipose-derived stem cells from Larimichthys crocea in different ratios was completed, it was found through immunofluorescence staining that a large number of myotubes were differentiated from the muscle satellite cells of Larimichthys crocea.

The use of the co-cultivation and co-differentiation method provided by the present disclosure can induce the myogenic differentiation of muscle satellite cells from Larimichthys crocea and the adipogenic differentiation of adipose-derived stem cells from Larimichthys crocea to promote an interaction between these two cells to allow a synergistic effect, such that cultivated meat has a close texture to an actual muscle tissue, which further improves a taste and flavor of the cultivated meat, allows the cell-cultivated meat to have balanced and healthy nutritional components, and meets the requirements of consumers for nutritional needs.

Unless otherwise specified, the raw materials and devices used in the present disclosure are all common raw materials and devices in the art. Unless otherwise specified, the methods used in the present disclosure all are conventional methods in the art.

The above is merely a preferred embodiment of the present disclosure and is not intended to limit the present disclosure in any way. Any simple modifications, changes, and equivalent transformations made to the above embodiment according to the technical essence of the present disclosure are within the protection scope of the technical solutions of the present disclosure.

What is claimed is:

1. A method for a co-cultivation and a directed differentiation induction of muscle satellite cells and adipose-derived stem cells, comprising the following steps:
(1) the co-cultivation: resuspending the muscle satellite cells and the adipose-derived stem cells from a Larimichthys crocea with a Larimichthys crocea stem cell complete medium, mixing, plating, and cultivating in a biochemical incubator at 26° C. to 30° C., wherein the Larimichthys crocea stem cell complete medium is changed every two days; and
(2) a co-differentiation: when the muscle satellite cells and the adipose-derived stem cells from the Larimichthys crocea co-cultivated in the step (1) grow to a density of 90% or more, removing the Larimichthys crocea stem cell complete medium, adding a Larimichthys crocea adipogenic differentiation medium to allow a differentiation for 7 d to 21 d, wherein the Larimichthys crocea adipogenic differentiation medium is changed every other day, and adding a Larimichthys crocea myogenic differentiation medium to allow a myogenic differentiation for 4 d to 6 d, wherein the co-differentiation lasts for 11 d to 27 d in total.

2. The method according to claim 1, wherein in the step (1), the Larimichthys crocea stem cell complete medium comprises a 90% DMEM/F12 basal medium and a 10% fetal bovine serum.

3. The method according to claim 1, wherein in the step (2), the Larimichthys crocea adipogenic differentiation medium comprises a 90% DMEM/F12 basal medium and a 10% fetal bovine serum, and further comprises the following adipogenic induction factors: insulin at a concentration of 1 mg/L to 10 mg/L, dexamethasone at a concentration of 0.5 μmol/L to 5 μmol/L, rosiglitazone at a concentration of 2 μmol/L to 20 μmol/L, indomethacin at a concentration of 10 μmol/L to 100 μmol/L, 3-isobutyl-1-methylxanthine at a concentration of 0.05 mmol/L to 1 mmol/L, arachidonic acid at a concentration of 1 μg/mL to 20 μg/mL, cholesterol at a concentration of 110 μg/L to 2,200 μg/L, tocopheryl acetate at a concentration of 35 μg/L to 700 μg/L, oleic acid at a concentration of 5 μg/L to 100 μg/L, linoleic acid at a concentration of 5 μg/L to 100 μg/L, palmitic acid at a concentration of 5 μg/L to 100 μg/L, stearic acid at a concentration of 5 μg/L to 100 μg/L, linolenic acid at a concentration of 5 μg/L to 100 μg/L, and myristic acid at a concentration of 5 μg/L to 100 μg/L.

4. The method according to claim 1, wherein in the step (2), the Larimichthys crocea myogenic differentiation medium comprises a 98% DMEM/F12 basal medium and a 2% horse serum, and further comprises the following myogenic induction factor: vitamin D at a concentration of 10 ng/mL to 300 ng/mL.

5. The method according to claim 1, wherein in the Larimichthys crocea stem cell complete medium and the Larimichthys crocea adipogenic differentiation medium, antibiotics are further added.

6. The method according to claim 1, wherein in the step (1), a process for isolating the muscle satellite cells and the adipose-derived stem cells from the Larimichthys crocea comprises the following steps:
(0-1) collecting a muscle tissue or an adipose tissue from the Larimichthys crocea, cutting the muscle tissue or the adipose tissue into pieces, and adding a digestion solution to allow a digestion for 0.2 h to 3 h to obtain a digested tissue mixed solution; adding a washing solution to the digested tissue mixed solution for a washing, filtering to obtain a first cell suspension, and centrifuging the first cell suspension at 800 rpm/min to 2,000 rpm/min for 5 min to 10 min to collect first centrifuged cells; and adding the washing solution to the first centrifuged cells for the washing, and centrifuging to collect second centrifuged cells;
(0-2) if visible red blood cells are observed in a cell pellet produced after a centrifugation, conducting a red blood cell lysis; and if no visible red blood cells are observed in the cell pellet produced after the centrifugation, directly proceeding to a next step; and
(0-3) resuspending the second centrifuged cells with the Larimichthys crocea stem cell complete medium to obtain a second cell suspension, inoculating the second cell suspension in a 6-well cell culture plate, and cultivating for 3 h to obtain a cell solution; transferring the cell solution to a new well, supplementing the Larimichthys crocea stem cell complete medium to 3 mL, and cultivating at 28° C.; and when a cell confluency reaches 85% to 90%, conducting a passage.

7. The method according to claim 6, wherein in the step (0-1), a mass-to-volume ratio of the muscle tissue or the adipose tissue to the digestion solution is 1:1 to 1:10, a mass-to-volume ratio of the muscle tissue or the adipose tissue to the washing solution is 1:1 to 1:10, a formula of the digestion solution comprises 0.1 mg/mL to 1 mg/mL of collagenase type I and 0.05 mg/mL to 1 mg/mL of trypsin, and the washing solution is a D-hanks solution.

8. The method according to claim 6, wherein the passage is conducted as follows: removing an old medium, adding 1 mL to 3 mL of phosphate-buffered saline (PBS) to wash cells from the 6-well cell culture plate 1 to 2 times to remove a residual serum, and adding 100 μL to 300 μL of 0.25% trypsin to digest the cells from the 6-well cell culture plate; when the cells from the 6-well cell culture plate are observed under a microscope to become spherical and most of the cells from the 6-well cell culture plate fall off after a tapping, adding 1 mL of the Larimichthys crocea stem cell complete medium to stop the digestion to obtain a third cell suspension, transferring the third cell suspension into a 2 mL Eppendorf tube and centrifuging the third cell suspension at 1200 rpm for 5 min, adding 2 mL of the Larimichthys crocea stem cell complete medium to resuspend cells after the third cell suspension is centrifuged, and gently pipetting up and down several times to obtain a fourth cell suspension; dispensing the fourth cell suspension into T25 flasks according to a volume ratio of 1:2 or 1:3, and supplementing a medium system in each of the T25 flasks to 5 mL; and sealing and labeling the T25 flasks, and incubating in the biochemical incubator at 28° C.
